(19) United States
(12) Patent Application Publication
(10) Pub. No.: US 2004/0049289 A1
(43) Pub. Date: Mar. 11, 2004

(54) Implant, method for inserting a reinforcing element, and method for producing an implant

(76) Inventors: Kornel Tordy, Oberbettingen (DE); Friedhelm Limbeck, Mechernich (DE); Janine Tordy, Oberbettingen (DE)

Correspondence Address: WILLIAM COLLARD, COLLARD & ROE, P.C., 1077 NORTHERN BOULEVARD, ROSLYN, NY, 11576, US

(21) Appl. No.: 10/450,688

(22) PCT Filed: Dec. 14, 2001
(86) PCT No.: PCT/DE01/04722

(30) Foreign Application Priority Data
Dec. 16, 2000 (DE) 10062691.2

(51) Int. Cl. A61F 2/02

(52) U.S. Cl. 623/23.76; 623/11.11

(57) ABSTRACT

To provide an implant that integrates as rapidly as possible with a tissue, and is nevertheless sufficiently compatible with the tissue, to invention suggests an implant made from a tissue-compatible, microporous material that has at least one stable opening.

The invention relates to an implant made from a tissue-compatible, microporous material, a process for the introduction of a reinforcing element into a soft structure surrounded by an enclosure and a process for the manufacture of an implant from a blank.

For example, nets, braided strips and cushions that may be implanted are known, particularly for surgical use. In this context, the materials that are used as a rule are polyamides or silicones. However, these have the disadvantage that they are not assimilated by the tissue adequately because of their configuration. This leads to incompatibilities such as capsule formations and even a rejection of the corresponding implant. The implants are mostly sewn onto the tissue, to prevent them from slipping. Particularly in the case of cushion implants, a further drawback consists in that the size of these implants must be determined and ordered before the surgical operation, disadvantageously, another operation becomes necessary if it becomes apparent during the operation that the intended implant does not match the required size. Since a surgical operation is always associated with risks during the procedure, particularly a second operation is a serious disadvantage.

On the other hand, implants made from a tissue-compatible, microporous material are sufficiently known from the state of the art and are offered for example in various variations for cosmetic surgery or accident surgery, e.g. as tissue replacement. In this field, particular use is made of expanded polytetrafluoroethylene (e-PTFE).

In such cases, it is disadvantageous that the period required by these known implants in order for cell tissue to grow into their microporous structure is too long, with the result that particularly in the event of an awkward movement the implant is often displaced from its intended position. In many cases, this also means that a further surgical operation becomes necessary, with the risks indicated above.

The invention is inspired by the task of providing an implant that forms a bond with a tissue as rapidly as possible, but still remains sufficiently compatible with the tissue.

The task of the invention is solved by an implant made from a tissue-compatible, microporous material that has at least one stable opening.

In an arrangement of such kind, a tissue that comes into contact with the implant can grow into the stable opening of the implant relatively quickly.

Advantageously, the tissue-compatible, microporous material includes labile openings. This is particularly advantageous since in addition to the stable openings, which aid the cell tissue in growing in rapidly, almost every area of the implant is suitable for enabling cell tissue to grow in, particularly in the medium and long terms.

This causes, besides a first, rapid fixing capability of the implant because of the cell tissue's quick growth into the stable opening, a particularly intimate bond between the tissue-compatible, microporous material of the implant and the cell tissue arranged around the implant.

It is also advantageous that no capsules are formed even with larger fillings, as is the case with silicone or almost all other solid implants. In addition, a considerable weight saving is achieved with the implant according to the invention as compared with the previously usual implants, such as silicone, sodium chloride, soya oil. With regard to the implant according to the invention, particularly leakage of the content of an implant is precluded. Thus the implant does not suffer damage with serious consequences as a result of pressure or low pressure. Moreover, the implant is practically indestructible as a result of puncturing or other wounds.

In order to assure particularly the tissue compatibility of the implant, it is advantageous if the implant is produced from an expanded polytetrafluoroethylene (e-PTFE). PTFE in particular has long been approved for use in surgery and to date has provoked no allergic reactions or incompatibilities or any kind.

Since an implant made from an e-PTFE is very soft and elastic, pressure points are advantageously created rarely if at all. This is particularly advantageous in areas such as for example nerves, bones, vessels, which particularly means that subsequent pain is all but eliminated.

With the expanded polytetrafluoroethylene a microporous is advantageously provided, the material of which is or may be expanded to such a degree that a tissue and/or vessel may grow through the microporous tissue itself, and in this way the implant enters into a very intimate bond with the tissue.

Particularly because of its porous structure, the microporous material made from an expanded polytetrafluoroethylene having stable openings has a ratio of advantageously just 14% material and 86% air.

In this way, the microporosity, or the labile openings, guarantee that in the medium and long terms tissue can penetrate the material and integrate with it. The implant with the stable openings is suitable for filling even the smallest tissue and/or node removals (both benign and malignant), which are clearly visible for example following an operation due to the depression in the skin because of an absence of tissue, since the implant may preferably be cut to any length and thus almost any missing tissue whose size and shape was not determined before a surgical operation, may be replaced. For example, a ball may be made from the implant, which may be inserted in place of the removed tissue. The implant, which itself has the form of a strip, and which is shaped into a ball or similar three-dimensional arrangement, thus forms a lattice structure, which may be replaced by tissue over time.

The implant according to the invention includes a monosubstance that preferably does not contain a filling, so that the disadvantage to which conventional implants are prone, that of outward diffusion of fluids, is advantageously avoided.

Of course, the implant according to the invention may be used as a support element or tissue replacement not only in human medicine, but also in veterinary medicine.

In order to establish an intimate bond between a tissue and the implant as quickly as possible by means of the stable openings, it is advantageous if at least one stable opening has a minimum diameter of 30 μm or 50 μm, preferably a minimum diameter of 1 mm that is essentially perpendicular to the implant surface. In this way, it is assured that a tissue grows into the microporous material of the implant particularly quickly.

In particular, the openings may be chosen in such a size that after the implant is inserted tissue or similar structures penetrate these openings so far that they come into contact again with native tissue or with other native structures. The adhesions brought about hereby lead directly to a stabilisation of the implant in the body. Further, the contact promotes the possible process of growing together that is then initiated significantly more quickly. In addition, it is conceivable that it—particularly where more stable or harder structures are concerned—that it may take somewhat longer until they themselves penetrate or grow into the relatively large openings. Nevertheless, this happens significantly more quickly than is the case with the smallest or labile openings.

However, in order particularly that sufficient implant material is available, it is advantageous if at least one stable opening has a diameter less than 5 mm, preferably a diameter equal to or less than 4 mm that is essentially perpendicular to the implant surface.

It is particularly advantageous if the stable openings of the microporous material, particularly in an implant that is under stress in the plane of the opening, are opened in such manner that their minimum diameters are greater than the minimum diameters of the labile openings. In this way, the danger is reduced that a recently inserted implant might be displaced by an awkward movement, or for example by a coughing fit, such that a repeat operation becomes necessary.

This distinguishes the implant according to the invention from known implants, since the pores of a known microporous material are blocked as a function of the stress of the implant or beforehand such that the ability of a tissue to grow in is severely hindered or all but impossible. This is caused, among other reasons, by the fact that individual fibres of the known implants become rigid in many stress conditions, so that the pores located respectively between them become constricted or blocked to such an extent that a tissue surrounding the implant can only grow into the pores with difficulty, and even then extremely slowly.

In contrast thereto, however, the stable openings of the implant according to the invention remain open under all usage-related stresses to such a degree that a tissue surrounding the microporous material may grow advantageously quickly into the stable openings and thus assures at least a basic attachment of the implant within the tissue as quickly as possible. In particular, under these conditions the danger of displacement soon after the surgical operation may be considerably reduced.

The size of the stable openings or the microstructure may be adapted within the spirit of the invention to the respective application, wherein the stable openings should be selected to be sufficiently large for a rapid penetration of the respective tissue and the microstructure sufficiently resilient for the required stresses and sufficiently open to allow long-term growing in of the tissue.

Of course, the stable openings may have practically any hole shape that may be created by technology. It is particularly advantageous if at least one stable opening has a circular or oval shape.

In addition, the stable openings may be arranged at any distance with respect to each other, wherein it has been found that it is advantageous if the stable openings have a separation of at least 0.5 mm, preferably a separation of at least 1 mm from each other.

Of course, the stable openings may be arranged in practically any pattern on the implant.

It is advantageous, particularly in the case of a vaginal strip, if the implant has a tubular or strip-shaped conformation. The implant is preferably conformed as an e-polytetrafluoroethylene strip. The implant may be varied at will with regard to its width, height or shape, wherein a rectangular form with 1 mm and 10 mm width is preferred.

Whereas in the case of a vaginal strip preferably an implant is used with attachment devices at both ends, an implant as tissue replacement may be provided preferably as a strip on a roll. Under these circumstances, an oval strip may be advantageous, since this provides volume in suitable manner. A rectangular strip is particularly suitable for manufacturing, being easier to process. For this reason, in the case of a strip as tissue replacement, a rectangular shape having 1.5 mm height and 4 mm width is preferred. In particular the implant in the form of a strip may be particularly conveniently inserted freely to a conglomerate through a small incision or through one or more small cuts, this being a method by which among other things a loose implant of any size may be produced.

One embodiment provides that the implant has at least one attachment device, by means of which a further component may be attached to the implant. Particularly in order to be able to guide the rather soft implant through a tissue, it is advantageous if the implant has an attachment device, to which for example a solid component may be affixed easily for guidance purposes.

Of course depending on the application the attachment device may be attached to the implant intraoperatively or by the manufacturer.

It is advantageous if the attachment device has means for positive attachment. In this way, a secure attachment may be established quickly.

In particular the attachment device may be furnished with a thread. In this way, it is ensured for example that practically any component may be attached to the implant quickly and easily. The component needs only to have a mating thread that matches the thread of the attachment device.

On the other hand, the attachment device may also have means for non-positive attachment.

The specialist in the art will recognise that the attachment device of the implant may be produced by a large number of technical arrangements, such as for example socket or clamping attachments. Of course, such an attachment device, particularly if attached to the implant by the manufacturer, independently of the other features of the invention, is advantageous for ensuring a secure insertion of an implant quickly and reliably.

A further embodiment provides that a guide means is attached to the attachment device, with which the implant may be guided particularly through a tissue. The guide means enables the implant to be guided advantageously through the tissue of the body without serious consequential injury, for example in a surgical operation to cure aconuresis. The guide means preferably has no perceptible and/or visible transition with respect to the attachment device of the implant.

To this end, the guidance means may be for example a blunt needle or a pilot pin. In particular, a blunt needle enables the implant to be inserted in the shortest possible time for example during an incontinence operation, whereby the danger of injury to tissue and/or vessels, for example injuries to the bladder and urethra is dramatically minimized.

In order to be able advantageously to guide the implant through the tissue, it is advantageous if the guide means is curved.

In order particularly to secure the guide means quickly and without difficulty to the implant, it is advantageous if the guide means has a coupling device that communicates with the attachment device. For example the attachment device of the implant has an external thread and the coupling device of the guide means has an internal thread corresponding with the external thread, so that the guide means may be secured particularly easily to the implant.

In order among other things that the danger of injury to the tissue may be further reduced, it is advantageous if the guide means is concavely curved, preferably spheroidal at the end opposing the coupling device. By a relatively rough structuring of this end, the tissue is damaged only within a reasonable zone. In the present context, the average radius of curvature of the tip should not be less than 50μ, preferably greater than 100μ or 300μ, in order to avoid injury to the extent possible. In this context, the average radius of curvature is understood to mean a radius of a semicircle that may be drawn from one side of the tip, extending to the tip and then to the other side, in which not only both sides but also the tip itself lie on this semicircle, while the remaining parts of the tip are arranged outside or on this semicircle.

The concavely curved end is preferably polished to minimise the danger of injuries.

The danger of damage to tissue or vessels may be further minimised if the coupling device of the guide means and the attachment device of the implant have an identical external diameter. In this way, particularly an attachment almost without transition may be created between the guide means and the implant. Among other things, this renders the guidance of the implant according to the invention through a body considerably easier, since no transitions are present between the guide means and the implant to impede guidance through a tissue. Moreover, the use of a protective tube or a protective sleeve between the implant and a tissue that is being punctured may be dispensed with.

In order to pass the guide means simply but gently through a body or through tissue, it is advantageous if the guide means is furnished with a vibration device. In consequence of such, extreme pressure does not need to be exerted on the guide means, as previously, to move this through the body, but the implant rather moves through the tissue of the body practically under its own power due to the vibrations of the guide means. Among other things, the danger of injuring internal organs, such as for example a bladder or of blood vessels, is further reduced hereby. In addition, the operating time is shortened significantly. For example in an operation for inserting a vaginal strip, the implant may be inserted in less than 20 minutes, possibly even under local anaesthetic.

Of course the vibration device may be attached to the guidance means externally and may be disposed internally in the guidance means. In addition, the vibration device may cause the guidance means to vibrate directly or by means of a flexible extension.

The vibration device is preferably arranged in the area of the coupling device or in the vicinity of the coupling device of the guide means, so that the latter may be inserted without difficulty.

In order to reduce overall costs, it is advantageous if the guide means is constructed in such manner that it may be inserted a second time, at least after a first insertion.

The attachment device and/or the guide means and/or the coupling device of the guide means is preferably made from V4A. In this way, their operating lives are prolonged as far as possible and they are easily sterilisable. In addition, this material has already proven to be ideally suited for use in medical equipment.

Of course, the devices and means named in the aforegoing may also be manufactured from other substances. This is particularly true if these substances also meet the stringent medical standards.

It is possible particularly during insertion of the implant described previously to dispense with the tubular and plastic sleeves required formerly for drawing the implant into a tissue. Among other things, this also ensures that the implant lies in particularly close proximity with the tissue to be punctured, which enables faster growing in of the tissue into the microporous material of the implant. Moreover, only a relatively small scar is left on the skin at a puncture point, which corresponds approximately to the diameter of the guide means, since because of the identical external diameter and due to the absence of the tubular sleeve or plastic sleeve the incising cut does not need to be unnecessarily enlarged. Among other things, this reduces the traumatic pain of a patient.

Of course, both the previously described guide means and the vibration means and/or the coupling between guide means and implant are advantageous even without the other features of the invention.

In the sense of the invention, the term “labile opening” is understood to mean those pores of the microporous material that under the effects of an application-related stress on the microporous material in the worst case close completely or to such a degree that at least temporarily no cell tissue can grow into the labile openings.

In the sense of the invention, the term “stable opening” is understood to mean those pores of the microporous material that under the effects of an application-related stress on the microporous material even in a severe case of introduced stress retain a minimum diameter, so that a cell tissue may penetrate and grow into the stable openings at almost all times.

In order to be able to manufacture a microporous material such as described in the preceding particularly easily, it is advantageous if the microporous material is an expanded polytetrafluoroethylene (e-PTFE). Particularly with an e-PTFE material, the fibrils are able to be expanded almost to any degree thus enabling a variously structured microporous material to be produced simply and inexpensively. In addition, e-PTFE has to date been entirely without pathological findings particularly in terms of medical uses with regard to incompatibilities or allergies.

The task of the invention is also solved by a procedure for the insertion of a reinforcing element into a soft structure surrounded by an enclosure, in which the reinforcing element is guided into the soft structure starting from a first area of the enclosure, the reinforcing element is guided through the soft structure as far as a second area of the enclosure and there is guided outwards, and in which the enclosure is made capable from the outside of allowing the reinforcing element to pass through in the second area, and the reinforcing element is then guided outwards through the second area.

It is advantageous is the reinforcing element is located from the outside in the second area of the enclosure beforehand, so that the enclosure may be opened efficiently at this position.

It is particularly advantageous if a part of the reinforcing element is reflected on the second area for purposes of making the enclosure capable of allowing the reinforcing element to pass through. The fact that the reinforcing element or at least a part thereof is reflected on the enclosure makes it possible to determine very easily from the outside at which site of the enclosure the reinforcing element is located. In this way, the site of the enclosure may efficiently be made capable of allowing the reinforcing element to pass through.

The procedure is suitable for example with regard to use in conjunction with aerosols surrounded by an enclosure. Other soft materials that are surrounded by a harder enclosure and that are to be reinforced by a reinforcing element may also be handled in this way. This procedure is also particularly suitable for drawing an implant into a body. In this case, the enclosure or the membrane is initially opened at a first site in such manner that the reinforcing element or an implant, if necessary with the aid of the guide means, is guided into the material or the body, and is advanced therethrough until the enclosure or the membrane is reached at a second site.

There, the reinforcing means, the implant and the guide means may be located and the enclosure or the membrane may be opened again from the outside, for example by an incision.

Further, the task of the invention is solved by a process for manufacturing an implant from a blank, wherein the blank is shaped as an implant intraoperatively. Since for example it is almost never possible to determine the extent of tissue removal that will be required before an operation, in future it will be possible to adapt the implant to the extent of the tissue removal directly intraoperatively.

It is particularly advantageous if the blank is cut to length for an implant intraoperatively. Particularly directly during an operation, for example for insertion of a tissue replacement, it is advantageous if the implant can be adapted exactly to the operating conditions by having the blank cut to size intraoperatively. In the case of a vaginal strip for example this may be done—possibly even after several days—if the implant is already inserted.

Further advantages, objectives and properties of the present invention are described in the description of the attached drawing, in which embodiments are represented in exemplary manner with regard to the implant according to the invention.

They show

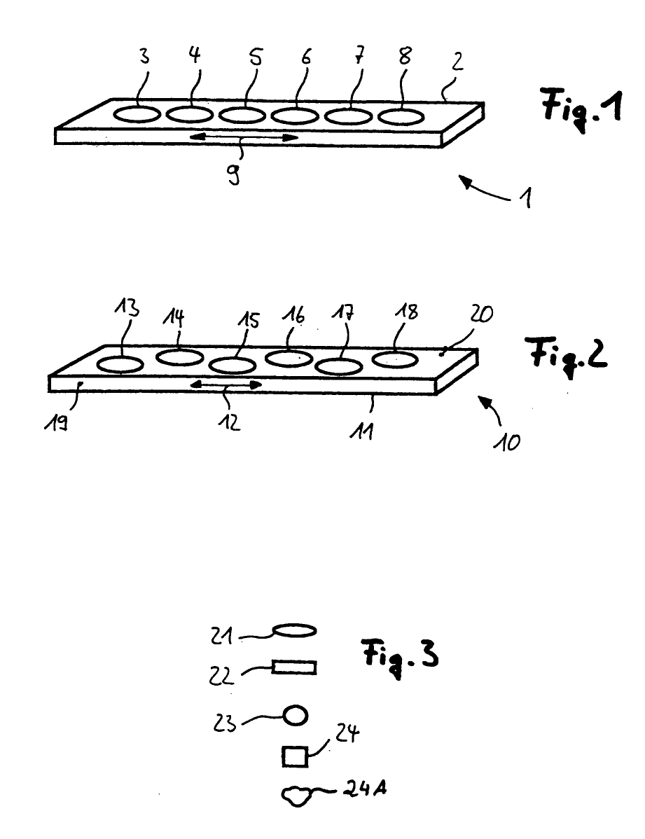

FIG. 1 a schematic view of a first arrangement variant of stable openings of an implant,

FIG. 2 a schematic view of a second arrangement variant of stable openings of an implant,

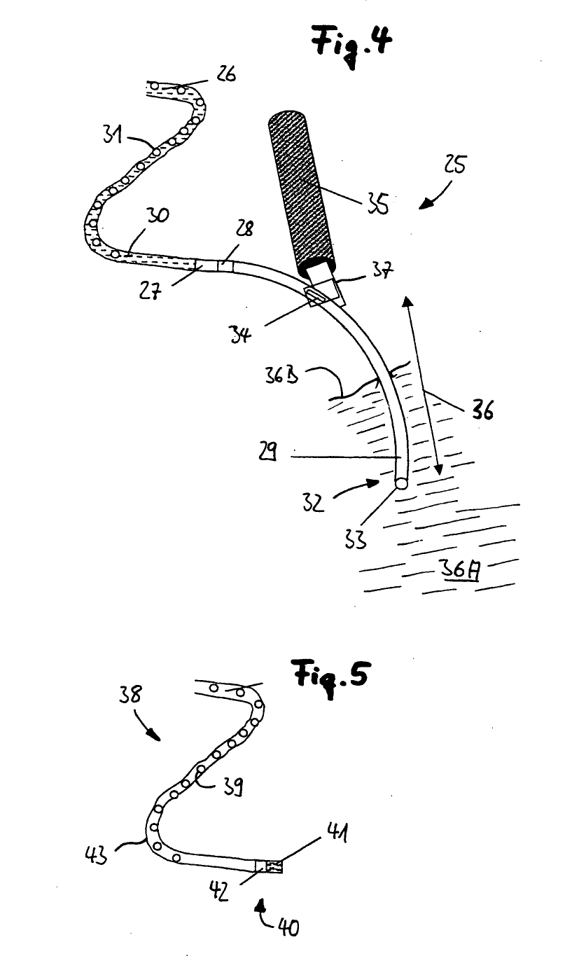

FIG. 3 schematically cross-sections of several implants,

FIG. 4 a schematic representation of an implant with a guide means and a vibration means,

FIG. 5 a schematic representation of a further implant with a connection device.

Implant 1 shown in FIG. 1 is made from a flat strip 2. In this case, flat strip 2 includes a plurality of holes 3, 4, 5, 6, 7 and 8 that are introduced into implant 1 as stable openings. In this case, holes 3 to 8 are each arranged at essentially equal distances from each other along a lengthwise extension 9 of flat strip 2.

In contrast with the above, implant 10 has on its flat strip-shaped body 11 a plurality of holes 13, 14, 15, 16, 17 and 18 that are offset with respect to one another along a lengthwise extension 12. Whereas holes 13 to 18 are also arranged in lengthwise extension 12 on body 11 of implant 10, yet holes 13, 15 and 17 are arranged closer to a lateral area 19 of implant 10, whereas holes 14, 16 and 18 are arranged rather in a lateral area 20 of implant 10.

In FIG. 3, cross-sections 21, 22, 23, 24 and 24 A of various implants are represented. In this case, cross-section 21 corresponds to an oval strip, cross-section 22 corresponds to a flat strip. Cross-section 23 has a circular conformation, while cross-section 24 is conformed as a rectangle, particularly a square. Reference number 24 A indicates a tube. This may either be constructed either internally rigidly or intrinsically labile.

The implant 25 illustrated in FIG. 4 includes an expanded polytetrafluoroethylene strip 26, which is disposed on an internal thread 28 of a curved needle 29 with a stainless steel screw 27.

Expanded polytetrafluoroethylene strip 26 has a plurality of labile openings 30 (only numbered for exemplary purposes here) and a plurality of stable openings 31 (also numbered for exemplary purposes only).

Curved needle 29 includes in the area of its tip 32 a polished, spheroidal tip 33. The rounded tip that has an average radius of curvature greater than 50μ, preferably greater than 300μ, is considered to be the tip.

Implant 25 and its curved needle 29 has a vibrator 35 in an region 34, which is preferably arranged in the vicinity of internal thread 28. By means of vibrator 35, needle 29 begins to oscillate, particularly in the area of polished spheroidal tip 33 in such manner that implant 25 may be advantageously moved in the direction of advance 36 through a tissue 36A. In order to reach tissue 36A with implant 25, a membrane 36B is—possibly—punctured that is disposed about tissue 36A. This may be done according to known methods, for example with a small cut, so that a needle that is itself capable of cutting the membrane may be dispensed with. Accordingly, the guide means and implant 25 may then be withdrawn again by guiding it as far as membrane 36B and located there. The guide means and implant 25 may then be withdrawn with a small cut.

Vibrator 35 transfers vibrations to needle 29 of implant 25 by means of an extension 37.

Implant 38 (FIG. 5) includes a plurality of stable openings 39 (numbered for exemplary purposes only here) and at one end 40 a screw 41 that is clamped together at its area 42 with a polytetrafluoroethylene strip 43 of implant 38.

1. Implant (1; 10; 25; 38) made from a tissue-compatible, microporous material, characterized by at least one stable opening (31; 39).

2. Implant (1; 10; 25; 38) according to claim 1, characterized in that the tissue-compatible, microporous material has labile openings (30).

3. Implant (1; 10; 25; 38) according to either of claims 1 or 2, characterized in that the implant (1; 10; 25; 38) is manufactured from an expanded polytetrafluoroethylene (e-PTFE).

4. Implant (1; 10; 25; 38) according to any of claims 1 to 3, characterized in that at least one stable opening (31; 39) has a minimum diameter of 30 μm or 50 μm, preferably a minimum diameter of 1 mm essentially perpendicular to the surface of the implant.

5. Implant (1; 10; 25; 38) according to any of claims 1 to 4, characterized in that at least one stable opening (31; 39) has a diameter of less than 5 mm, preferably less than or equal to 3 mm essentially perpendicular to the surface of the implant.

6. Implant (1; 10; 25; 38) according to any of claims 1 to 5, characterized in that the stable openings (31; 39) opposite the labile openings (30), particularly in an implant (1; 10; 25; 38) under stress in the plane of the openings, are opened in such manner that their minimum diameters are greater than the minimum diameters of the labile openings (30).

7. Implant (1; 10; 25; 38) according to any of claims 1 to 6, characterized in that at least one stable opening (31; 39) has a circular or oval shape.

8. Implant (1; 10; 25; 38) according to any of claims 1 to 7, characterized in that the stable openings (31; 39) are at a distance of at least 0.5 mm, preferably a distance of at least 1 mm from each other.

9. Implant (1; 10; 25; 38) according to any of claims 1 to 8, characterized in that the implant (1; 10; 25; 38) has a tubular or strip conformation.

10. Implant (1; 10; 25; 38) according to any of claims 1 to 9, characterized in that the implant (1; 10; 25; 38) has at least one attachment device, by means of which a further component can be attached to the implant.

11. Implant (1; 10; 25; 38) according to claim 10, characterized in that the attachment device has means for positive attachment.

12. Implant (1; 10; 25; 38) according to either of claims 10 or 11, characterized in that the attachment device has a thread.

13. Implant (1; 10; 25; 38) according to claim 10, characterized in that the attachment device has means for non-positive attachment.

14. Implant (1; 10; 25; 38) according to any of claims 10 to 13, characterized in that a guide means is connected to the implant (1; 10; 25; 38) by means of the attachment device, which guide means can be used to guide the implant particularly through a tissue.

15. Implant (1; 10; 25; 38) according to claim 14, characterized in that the guide means is concavely curved, preferably spheroidal, at the end facing the coupling device.

16. Implant (1; 10; 25; 38) according to claim 15, characterized in that the concavely curved end is polished.

17. Implant (1; 10; 25; 38) according to any of claims 14 to 16, characterized in that the guide means is a blunt needle (29).

18. Implant (1; 10; 25; 38) according to any of claims 14 to 17, characterized in that the guide means is curved.

19. Implant (1; 10; 25; 38) according to any of claims 14 to 18, characterized in that the guide means has a coupling device that communicates with the attachment device of the implant (1; 10; 25; 38).

20. Implant (1; 10; 25; 38) according to claim 19, characterized in that the coupling device of the guide means and the attachment device of the implant have identical external diameters.

21. Implant (1; 10; 25; 38) according to any of claims 14 to 20, characterized in that the guide means has a vibration device.

22. Implant (1; 10; 25; 38) according to any of claims 1 to 21, characterized in that the microporous material consists of a plurality of fibrils, wherein the fibrils are connected to each other in groups in the area of their extremities.

23. Implant according to claim 22, characterized in that the fibrils form labile openings greater than 30 μm or 50 μm.

24. Procedure for the insertion of a reinforcing element into a soft structure surrounded by an enclosure, in which the reinforcing element is guided into the soft structure starting from a first area of the enclosure, through the soft structure as far as a second area of the enclosure and there is guided outwards, characterized in that the enclosure is made capable from the outside of allowing the reinforcing element to pass through in the second area, and the reinforcing element is then guided outwards through the second area.

25. Insertion procedure according to claim 24, characterized in that the reinforcing element is located in the second area of the enclosure beforehand from outside.

26. Insertion procedure according to either of claims 24 or 25, characterized in that in order to provide the ability to allow the reinforcing element to pass through a part of the reinforcing element is reflected on the second area.

27. Process for manufacturing an implant (1; 10; 25; 38) from a blank, characterized in that the blank is formed into an implant (1; 10; 25; 38) intraoperatively.

28. Manufacturing process according to claim 27, characterized in that the blank is cut to length intraoperatively.
